(19) United States
(12) Patent Application Publication
(10) Pub. No.: US 2024/0027171 A1
(43) Pub. Date: Jan. 25, 2024

(54) HAND HELD ANTI-ASSAULT WEAPON

(71) Applicant: Robert WOOD, Fort Washington, MD (US)

(72) Inventor: Robert WOOD, Fort Washington, MD (US)

(21) Appl. No.: 17/870,322

(22) Filed: Jul. 21, 2022

(51) Int. Cl. F41H 13/00; G01N 1/04; A62B 3/00

(52) U.S. Cl. CPC F41H 13/0018; G01N 1/04; A62B 3/00

(57) ABSTRACT

A compact, hand-held anti-assault weapon for repelling attackers while avoiding serious injury to the potential victim. Weapon includes a rigid, metallic, cylindrical body with a concave section to serve as a secure hand grip. End caps seal the opposite ends of the body and define the hollow interior of the body. An electroshock generator is housed within the cylindrical body, and a button, on the exterior of the body, is depressed to actuate the electroshock mechanism, such as a stun gun, or Tazer. An ear on one end cap may be used to break glass, while a series of protrusions on the second end cap may be used to collect a DNA sample from an attacker. The electroshock generator discharges a burst of high intensity electrical beams through the second end cap to shock an attacker.

### BACKGROUND OF THE INVENTION

The invention pertains generally to hand-held anti-assault weapon for defeating a physical attack upon a person. More particularly, the invention pertains to a hand-held anti-assault weapon with a plurality of protrusions for raking over the face, arms, or other body parts of an attacker to collect a DNA specimen from the attacker, combined with a stun-gun mechanism for repelling the attacker.

Various anti-assault weapons that deter, or repel, attackers have been devised and implemented. Such weapons, to be effective, must inflict intense pain and/or discomfort upon the attacker to deter or discontinue an attack. Known anti-assault weapons rely upon electrical shocks administered to the attacker, or pepper sprayed in the face and eyes of the attacker. Other known anti-assault weapons are shaped as batons or clubs, and the weapon is swung to deliver sharp blows to the attacker.

Anti-assault weapons must be light in weight and easy to actuate, so that a relatively small, potential victim can punish a would-be attacker. Furthermore, the anti-assault weapon should be capable of storage in a small space, such as a woman's handbag, the glove box of a car, or in the pocket of an article of clothing, etc. Lastly, the anti-assault weapon must safely be maintained in an inoperative state, lest the power for delivering electrical shocks be prematurely discharged, and not available when an emergency arises.

### SUMMARY OF THE INVENTION

The invention relates generally to a relatively simple, hand-held anti-assault weapon that is versatile, effective for its intended purposes, is compact, yet sturdy and does not inadvertently discharge prior to actuation for its intended purpose.

The weapon employs a rigid, hollow body that is cylindrical in cross-section for most of its length, with end caps sealing the opposite ends of the body. A section of the body curves inwardly to form a hand grip between the pair of end caps. An ear is formed on a first end cap, and a passage is defined through the ear. A chain, cord, or lanyard passes through the ear, and keys and a whistle may be secured to the chain or lanyard. The ear may be used to smash through glass in the event that the individual carrying the anti-assault weapon is trapped, or retained, against his or her will, in the interior of an automobile or other vehicle, or in a room in a building. Such retention may be caused by a natural disaster or by a potential attacker.

A second, larger end cap, is located at, and secured to, the opposite end of the rigid body. The exposed face of the second end cap includes two unique mechanisms. The first mechanism is a series of protrusions, which can be drawn or raked over the face of an attacker, or any other exposed body part. The protrusions collect a DNA sample, which can be tested to identify the attacker, at a later date.

The second mechanism is an electroshock weapon, such as a stun gun or taser, which emits high intensity bursts of electrical energy through the protrusions.

A button resides in a shallow recess on the upper surface of the body of the anti-assault weapon. The button is normally retained in an inoperative condition by a spring, or other basing member. When the button is depressed, the electroshock mechanism, located within the body of the anti-assault weapon, is energized and delivers high intensity bursts of electric energy.

Snap rings are provided to retain the first and second end caps in fixed position atop the body the weapon. The end caps may be screwed into complementary threads on the interior of the body of the anti-assault weapons, or may be forced into locking engagement with the interior of the body.

The invention requires a minimum number of components, that are easy to manufacture and assemble, thereby leading to production of the anti-assault weapon at a reasonable price. The anti-assault weapon may be used in an instinctive manner, and may be included in the teaching of self defense and martial arts skills.

### BRIEF DESCRIPTION OF THE DRAWINGS

FIG. 1 is a first perspective view of applicant's anti-assault weapon, the view being taken from the base of the weapon looking toward the upper end;

FIG. 2 is a second perspective view of applicant's anti-assault weapon, the view being taken from the upper end of the anti-assault weapon looking toward the base;

FIG. 3 is a side elevational view of applicant's anti-assault weapon;

FIG. 4 is a top plan view of applicant's anti-assault weapon;

FIG. 5 is a plan view of the base of applicant's anti-assault weapon, such view being taken along line 5-5 in FIG. 4 and in the direction indicated; and

FIG. 6 is a plan view of the upper face of applicant's anti-assault weapon, such view being taken along line 6-6 in FIG. 4 and in the direction indicated.

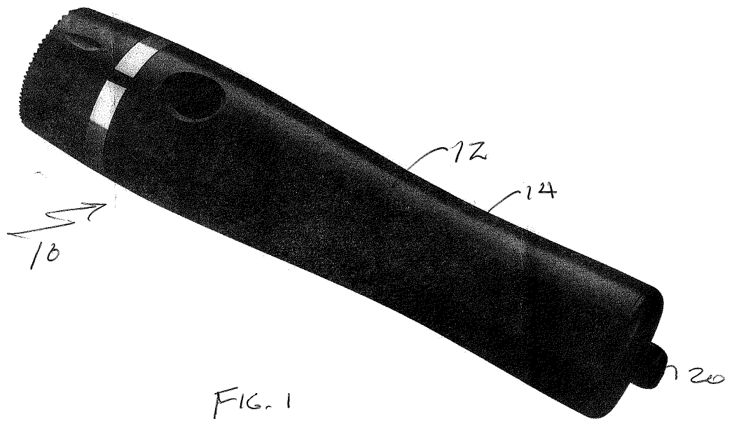

### DETAILED DESCRIPTION OF THE EMBODIMENTS

Applicant's unique anti-assault weapon is indicated generally by reference numeral 10. Weapon 10 includes a hollow, generally cylindrical body 12. A concave section 14, is defined in body 12 to provide a surface that can be readily grasped by a user. A first end cap 16 seals the base of the body 12, and second end cap 18 seals the opposite end or base, of body 12. Ear 20 projects outwardly from end cap 16; and aperture 22 extends through ear 20. A chain 24, a lanyard 24, or cord, or the like, is passed through aperture 22 in ear 20 to secure keys, a small flashlight, a whistle, etc. (not shown) to anti-assault weapon 10.

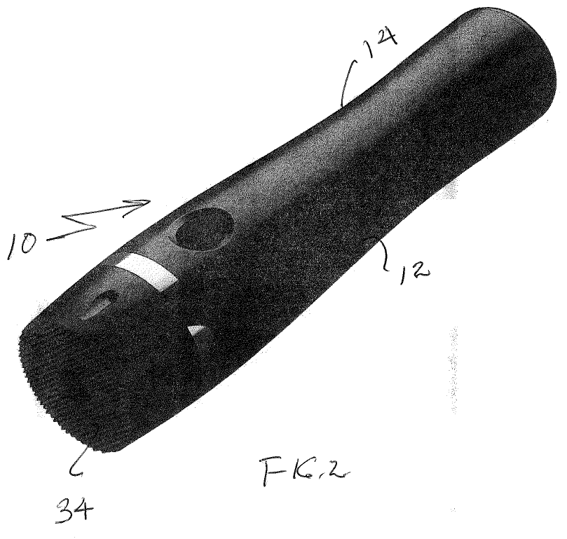

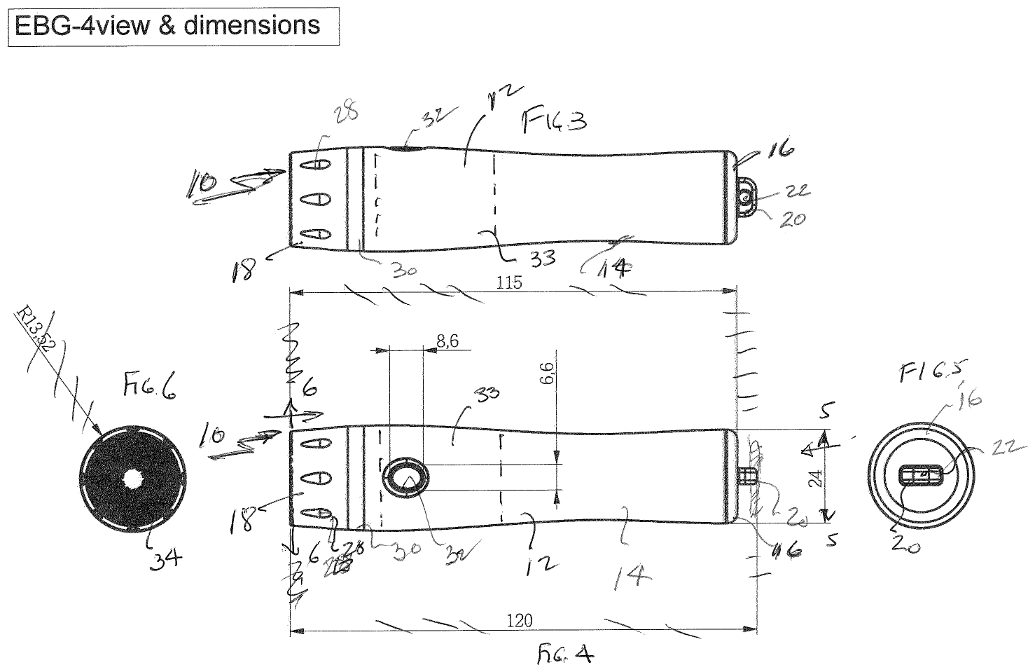

A second, larger end cap 26 seals the upper or opposite end of body 12. Protrusions 28 are formed on end cap 26. End cap may be screwed, pressed, or otherwise secured into sealing engagement with body 12. A power source (not shown) is retained in the interior of body 12 adjacent end cap 26 in fixed position. A depressible button 32 is located atop body 12 of anti-assault weapon 10. The button is normally based upwardly, to an inoperative position an electroshock assembly 33 is located in the interior of body 12, in proximity to button 32, and adjacent to a power source. Assembly 33 is shown in phantom outline FIGS. 3 and 4.

A plurality of sharp protrusions, 34 are distributed about the exposed face of end cap 18. The protrusions, when pressed against the exposed skin of a potential or actual attacker, and drawn across the skin, collect sufficient tissue to allow DNA testing to identify the attacker at a later date.

When necessary, button 32 is depressed to actuate electroshock assembly 33 which triggers the emission of bursts of high intensity electrical energy. The electrical energy is directed through the exposed face 36 of end cap 26. Known electroshock assemblies are sold as stun guns, or identified by the trademark, Tazer.

Thus, one can appreciate that applicant has devised a hand held anti-assault weapon 10 with the capacity to (1) repel an attacker, or deter a would be attacker, by the use of an electroshock mechanism, while (2) collecting a DNA sample from the attacker or potential attacker. Such desirable operating characteristics are achieved by a hand-held weapon that can be readily manufactured and easily maintained in operative condition, for an extended period of time. Additionally, the anti-assault weapon is easy to use without specialized training or demonstrated in martial arts, and is stored or retained in a woman's handbag, a man's pants pocket, or any pocket in a piece of apparel.

1. (canceled)

2. An anti-assault weapon as defined in claim 4 wherein said button is normally based to an inoperative position, thereby preventing inadvertent operation of said electroshock generator.

3. An anti-assault weapon as defined in claim 4 wherein the output of electrical pulses from said electroshock generator exits the body of the anti-assault weapon through the protrusions on said second end cap.

4. An anti-assault weapon comprising,
a) a rigid hollow cylindrical body adopted to fit in the hand of a user,
b) a first opening at one end of said body and a second opening at the opposite end of said body,
c) a first end cap to seal said first opening, against light passing therethrough,
d) an ear defined on the outer surface of said first end cap, said ear being adapted to smash through glass,
e) protrusions defined on the exposed outer face of said second end cap, said protrusions adapted to capture a DNA sample from a potential attacker;
f) an electroshock assembly located within the interior of said body of said weapon, and
g) a button situated on the exterior of said body, said button, when depressed, actuating said electroshock assembly to emit bursts of high intensity electrical energy through said second end cap to stun a potential attacker.
